(19) United States
(12) Patent Application Publication
(10) Pub. No.: US 2017/0035481 A1
(43) Pub. Date: Feb. 9, 2017

(54) DEVICES, SYSTEMS AND METHODS FOR USE IN BONE TISSUE

(71) Applicant: Woven Orthopedic Technologies, LLC, Manchester, CT (US)

(72) Inventors: Francis Patrick MAGEE, Mackay, ID (US); Robert LUZZI, Silverthorne, CO (US)

(73) Assignee: Woven Orthopedic Technologies, LLC, Manchester, CT (US)

(21) Appl. No.: 15/230,266

(22) Filed: Aug. 5, 2016

Related U.S. Application Data
(60) Provisional application No. 62/201,314, filed on Aug. 5, 2015.

(51) Int. Cl. A61B 17/84; A61B 17/88; A61B 17/86

(52) U.S. Cl. CPC A61B 17/842; A61B 17/86; A61B 17/88; A61B 2017/00853

(57) ABSTRACT

A woven retention device can include a pair of connected opposing lattice segments comprising intersecting filaments. The pair of connected opposing lattice segments can be configured to be placed in a portion of a circumference of a bone hole of a bone. A method of inserting a woven retention device can include inserting a woven retention device into a bone hole of a bone. The inserting the pair of opposing lattice segments can cover only a portion of a circumferential area of the bone hole. The method can include securing a fastening device into the bone hole having the inserted woven retention device. Upon securing the fastening device into the bone hole, a portion of the fastening device interfaces directly with the surface of the bone in the bone hole and a portion of the fastening device can interface directly with the bone interface of the woven retention device.

### CROSS-REFERENCE TO RELATED APPLICATIONS

This application claims priority to U.S. Provisional Patent Application No. 62/201,314, filed Aug. 5, 2015, which is hereby incorporated by reference herein in its entirety.

### TECHNICAL FIELD

The present invention relates to devices, systems and methods for use in fixing fasteners to bone tissue.

### BACKGROUND

In orthopedic surgery it is common to secure a bone screw to a patient's bone. Bone fracture repair is surgery to fix a broken bone using plates, nails, screws, or pins. It is common in the treatment of fractures to attach a plate to the bone utilizing bone screws. The resulting construct prevents motion of the fractured bone so that the bone can heal. Alternatively, one or more screws may be inserted across the break to hold it in place.

In the treatment of spinal disorders, pedicle screws are inserted into the patient's vertebrae to serve as anchor points that can then be connected with a rod. This construct prevents motion of the vertebral segments that are to be fused.

In the treatment of detached tendons, screw-like tissue anchors are inserted into the patient's bone to serve as an anchor for the reattachment of the tendon.

One complication with the use of bone screws is the loss of fixation or grip between the bone screw and the patient's bone. Another complication with the use of bone screws is the stripping of the hole in the bone when the bone screw is inserted. This results in the loss of purchase and holding strength of the bone screw.

The presence of osteoporotic bone can increase the likelihood of complications by reducing the purchase or grip of the bone screw to the patient's bone, resulting in a loss of holding strength and loosening of the bone screw or pullout of the bone screw.

Current solutions to secure bone screws have not adequately addressed screw failure and the underlying causes of screw failure. Also, current solutions have not adequately addressed screw failure related to bi-cortical intramedullary anchorage.

### BRIEF SUMMARY OF THE INVENTION

There is a need for devices, systems and methods that enhance the surface of a bone hole to provide enhanced fixation of a bone anchor to the bone. Additionally, there is a need for devices, systems and methods for repairing the surface of the bone hole following damage to the bone hole as in the case of stripping of the hole in the bone when a bone screw is over-tightened. Also, there is a need for devices, systems and methods for providing an enhanced bone hole surface for the reattachment of tendons in, for example anterior/posterior cruciate ligament repair procedures, rotator cuff repair procedures, etc. There is a need for a device that enhances the surface of a bone hole to enhance fixation of a bone anchor to bone and permits bone ingrowth into its structure. There is a need for a single device that enhances the surface of a bone hole to enhance fixation of a bone anchor to bone and accommodates variations in the diameter and depth of the bone hole. Further, there is a need for such devices that have enhanced biocompatibility to aid in tissue and bone healing, regeneration, and growth.

A woven retention device can include a pair of connected opposing lattice segments comprising intersecting filaments. The pair of connected opposing lattice segments can have a size and the size can be configured for the opposing lattice segments to be placed in a portion of a circumference of a bone hole.

The pair of opposing lattice segments can be connected at a connecting edge joining the pair of lattice segments on at least a portion of at least one edge.

The woven retention device can further include a bone interface on an exterior surface of one of the lattice segments that is configured to interface with a bone surface of a bone hole. The woven retention device can further include a fastening device interface on an exterior surface of an other of the lattice segments that is configured to interface with a fastening device.

The lattice of intersecting fibers can include braid patterns that vary protrusion locations of the bone interface and create an asymmetrical pattern to engage a surface of the bone hole.

When the woven retention device is inserted into the bone hole, the woven retention device can cover in a range of about ⅓ to about ⅛ of the circumference of the bone hole.

The fastening device can be a screw and the other of the lattice segments can be formed of a material that does not cut when interfaced with the screw.

The pair of opposing segments can be lubricious and compliant to embed into bone and adapted to allow the screw to slide past/thread without cutting the lattice segments.

The bone interface can interface with the bone by interdigitation and/or microloading.

The pair of opposing lattice segments can include a continuous lattice of intersecting fibers.

The pair of connected opposing lattice segments can include a flattened woven sleeve.

The woven retention device can include two folds in the continuous lattice.

The pair of opposing lattice segments can include two distinct layers that are coupled on at least one end.

A proximal portion of the woven retention device can be flattened and a distal portion of the woven retention device can be cylindrical. The fastening device can interface with an exterior surface of the proximal portion and the fastening device can interface with an interior surface of the distal portion.

At least one of the lattice segments can be configured to slide with respect to the corresponding opposing lattice segment.

A method of using a woven retention device can include inserting a woven retention device into a bone hole of a bone. The woven retention device can include a pair of opposing lattice segments, a connecting edge joining the pair of lattice segments on at least a portion of at least one edge, and a bone interface on an exterior surface of one of the lattice segments. Inserting the pair of opposing lattice segments can cover only a portion of a circumferential area of the bone hole. The method can include securing a fastening device into the bone hole having the inserted woven retention device. Upon securing the fastening device into the bone hole, a portion of the fastening device can interface directly with the surface of the bone in the bone hole and a portion of the fastening device can interface directly with the bone interface of the woven retention device.

The method can further include inserting a plurality of woven retention devices into the bone hole.

The opposing lattice segments of the inserted woven retention device can provide resistance to rotation of the inserted woven retention device in contact with the fastening device.

The method can include inserting the woven retention device to just past a distal cortex region of the bone, inserting the woven retention device to the distal cortex of the bone, and securing the fastening device at a point that corresponds to a start of the insertion at a proximal cortex of the bone.

Upon the fastening device being secured, the woven retention device can cover ⅓ to about ⅛ of a circumference of the bone of the bone hole.

The woven retention device can include a distal portion that is cylindrical and a proximal portion that is flattened. The fastening device can include inserting the fastening device into a cylindrical area of the distal portion and abutting the fastening device against the exterior surface of the proximal portion of the woven retention device.

Additional features, advantages, and embodiments of the invention are set forth or apparent from consideration of the following detailed description, drawings and claims. Moreover, it is to be understood that both the foregoing summary of the invention and the following detailed description are examples and are intended to provide further explanation without limiting the scope of the invention as claimed.

### BRIEF DESCRIPTION OF THE DRAWINGS

FIG. 1 illustrates a flattened woven retention device, according to an embodiment of the invention.

FIG. 2A illustrates the insertion of the flattened retention device in a bone hole covering only a portion of the circumference of the hole, according to an embodiment of the invention.

FIG. 2B shows the screw engaged in the bone and the retention device under slight traction to one side, according to an embodiment of the invention.

FIG. 3 demonstrates the fully inserted screw and retention device captured between the bone and the screw on only a portion of the circumference, according to an embodiment of the invention.

FIG. 4A-D illustrate insertion of the screw and mating with the flattened retention device, according to an embodiment of the invention.

FIG. 5 is a perspective view of a woven retention device, according to an embodiment of the invention.

FIG. 6 is a side view of FIG. 5.

FIG. 7 is an elevated view of FIG. 5.

FIG. 8 is a cross-sectional view of FIG. 6.

### DETAILED DESCRIPTION OF THE INVENTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

According to an embodiment of the present invention, a woven retention device can be inserted into a pilot hole and a screw can be inserted to abut the woven retention device. In this embodiment, a face of the screw makes contact with a flattened or folded-over woven retention device while a face of the screw makes contact directly with the bone. A same amount (volume) of material can be placed inside the pilot hole which should lead to a similar or possibly improved increase pull-out resistance compared to a standard screw.

According to an embodiment of the present invention, the level of the material of the woven retention device above the bone surface can be very important. If the level of the woven retention device is too deep then the screw may not find the lumen and/or may push the woven retention device with the screw as the screw proceeds into the lumen. On the other hand, if the woven retention device is too proud, there may be difficulty engaging bone, there may be fiber disruption, or there may be debris formation. Another challenge lies in the general difficulty in engaging bone with the interposition of the woven retention device. For example, a diameter mismatch may occur between the pilot hole and the screw (2.5 mm vs 3.5 mm).

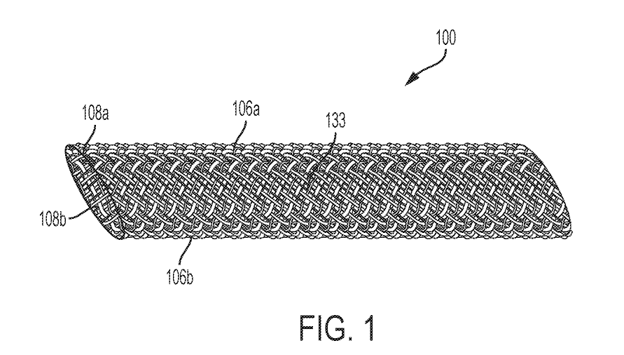

As shown in FIG. 1, a woven retention device 100 can include a pair of opposing lattice segments 108a, 108b comprising intersecting fibers. The intersecting fibers can include braid patterns that vary protrusion locations of the bone interface and create an asymmetrical pattern to engage a surface of the bone hole. The fibers can be arranged as described in U.S. application Ser. No. 14/569,542, the content of which is hereby incorporated by reference herein in its entirety.

At least one of the lattice segments is configured to slide with respect to the corresponding opposing lattice segment. The woven retention device can include a connecting edge 106a, 106b joining the pair of lattice segments 108a, 108b on at least a portion of at least one edge 106a, 106b. The woven retention device 100 can include a bone interface 107 on an exterior surface of one of the lattice segments 108a.

The pair of opposing lattice segments can include a continuous lattice of intersecting fibers. The woven retention device can include two folds in the continuous lattice. The pair of opposing lattice segments can include two distinct layers that are fastened together on at least one end. Mechanical mechanisms by which the segments can be attached can include by suture, sewn together, adhesive, etc.

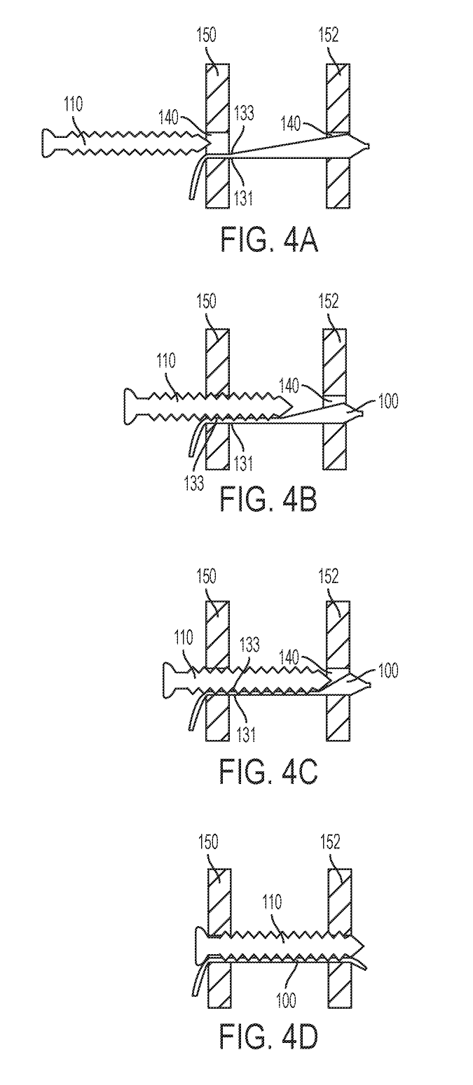

The lattice of intersecting fibers can include braid patterns that vary protrusion locations of the bone interface and create an asymmetrical pattern to engage the bone surface. Thus, as can be seen in FIG. 4A, the woven retention device 100 can include a bone interface 131 on an exterior surface of one of the lattice segments that is configured to interface with a bone surface of a bone hole. The bone interface can interface with the bone by interdigitation and/or microloading. The interdigitation mechanism and microloading mechanisms is described, for example, in U.S. Pat. No. 8,956,394, the content of which is hereby incorporated by reference herein in its entirety.

The woven retention device can include a fastening device interface 133 on an exterior surface of an other of the lattice segments (that can be different from the one lattice segment that has the bone interface) that is configured to interface with a fastening device 110. In an embodiment where the fastening device is a screw, the other of the lattice segments is formed of a material that does not cut when interfaced with the screw.

When the woven retention device 100 is inserted into the bone hole, the woven retention device can cover less than the full circumference, preferably in a range of about ⅓ to about ⅛ of the circumference of the bone hole.

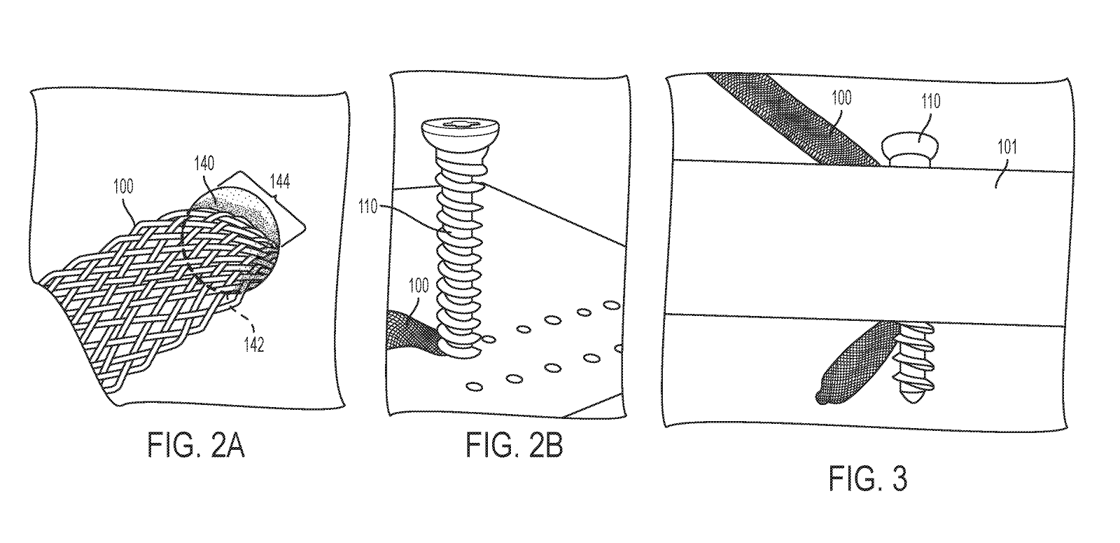

FIGS. 2A and 2B show that while the woven retention device is in the pilot hole, there is room for a fastening device 110 to be started and driven into the pilot hole while maintaining light traction on the tail of the woven retention device. For example, in FIG. 2A a woven retention device 100 can be draped into a pilot hole 140, where the portion of the woven retention device 100 that contacts the edge of the pilot hole is depicted as 142, and where the portion of the pilot hole 140 that does not contact the woven retention device 100 is depicted as 144. As shown in FIG. 2B, the fastening device 110 can be a screw. The screw can be a self-tapping screw that is configured to tap its own hole as it is driven into the bone. FIG. 2B shows the screw being inserted into the bone hole after the sleeve of FIG. 2A is fully inserted into the hole.

The pair of opposing segments can be lubricious and compliant to embed into bone and adapted to allow the screw to slide past or thread without cutting the pair of opposing lattice segments. That is, the pair of opposing segments can be smooth and/or slippery with or without a lubricant.

FIG. 3 shows the screw inserted into the bone 101 and the positioning of the woven sleeve pressed between one side of the pilot hole and the fastening device. FIG. 3 shows the screw fully inserted where the woven retention device covers approximately ⅓ of the circumference of the screw/bone interface. The coverage can be significantly less than the full circumference and still provide adequate bone engagement for increased axial pull-out strength. In a preferred embodiment, one quarter of the circumference is covered. In another embodiment, as little as ⅛ of the circumference is covered. In an embodiment, a parameter to the effectiveness of the flattened strip is the amount of material that is wedged between the bone 101 and the screw (thickness) radially for a given screw diameter to bone hole diameter, not the circumference coverage (width). For a given clinical application, the location of the radial coverage can be optimized to improve safety and/or effectiveness. For example, in the spine where there is concern over a pedicle breach into the spinal canal anteriorly, the flattened material can be positioned appropriately.

As the fastener advances in the hole, the friction of the screw with the woven retention device is less than the woven retention device to the bone and thus the screw advances without dragging the sleeve further into the hole. The flattened sleeve configuration can enhance the insertion of a fastener into a bone hole by creating an interface of two opposing woven retention device surfaces which can slide past each other. The retention device's fiber braiding or weave pattern creates a track where the force to advance in the hole is less than the force to move laterally thus guiding the screw into the hole. In an embodiment, the braid can create a helical track that matches the helical thread of the screw.

A woven retention device can be reduced in diameter and inserted into a pilot hole (PH) that spans a near or proximal cortex 150 and a far or distal cortex 152. In between the near cortex 150 and the far cortex 152, there is no intramedullary bone in one embodiment. A self-tapping screw can be inserted into the already inserted woven retention device. The screw upon entering the woven retention device can dilate a portion of the woven retention device at or close to its natural at-rest diameter. As the screw continues to advance to the end of the woven retention device, the woven retention device continues to dilate to fit. As the screw approaches a far or near cortex or inner cortex bone, an area of a woven retention device becomes susceptible to breakage or damage as the screw and the bone can pinch or put pressure on a portion of the woven retention device.

FIGS. 4A-D show a woven retention device being inserted into a pilot hole. As shown, the woven retention device is flattened or compressed laterally into a strip against a wall of the pilot hole. Thus the flattened strip of the retention device can be a flattened tube as shown in FIGS. 1-4.

Alternatively, the flattened strip can be made of one piece of any of the numerous two-dimensional weave or fabric geometries that are known in the art. In an embodiment, the piece can be folded or rolled, creating two or more layers where one layer slides with respect to the other. An advantage of having a folded end is eliminating any free fiber ends as well as providing resistance to rotation of the layer in contact with the screw. Alternatively, the flattened strip can be made of 2 distinct layers with each layer is then interlocked or fastened together on at least one end. The layers of flattened strips may comprise a different braid or weave patterns in order to vary the protrusion locations and thus create an asymmetrical pattern to engage the bone surface. The flattened strip can be made from solid porous material, made from one of numerous lubricious compliant materials. In one preferred embodiment, the coefficient of friction of the material is less than 0.2 In a preferred embodiment, the hardness is less than Rockwell M94. Alternatively, the flattened strip may be composed of a flattened tube for a portion of the length and a portion may be only one layer.

The woven retention device can be flattened in a bean shape, as shown in FIG. 3, after the insertion of the screw. At this point, a cutting device could be designed to cut off the device prior to the final insertion of the screw.

FIGS. 4A-4D show a side view of the screw being fully inserted into the pilot hole 140 with the woven retention device 100 to the side. In one embodiment, the woven retention device 100 is inserted to just past the distal cortex 152. FIG. 4A shows the flattened strip inserted to the distal cortex 152 and the screw 110 at the start of the insertion at the proximal cortex 150. FIGS. 4B and 4C illustrate how the screw 110 flattens the retention device 100 as it advances distally. FIG. 4D shows the fully inserted screw and a completely flattened retention device.

Although only one retention strip is shown in FIGS. 4A-4D, it is envisioned that multiple strips can be inserted at various locations along the circumference of the bone hole. For example, four strips at 90 degrees from each other on the circumference is only one of numerous examples. Alternatively, FIGS. 4A-4D illustrate the condition when the flattened retention device is inserted to the end of the hole at the distal cortex 152 in a long bone. There may be other clinical applications that would benefit from insertion of the strip only part way in the hole, such as at the proximal end only.

Thus, a method of using a woven retention device can include inserting a woven retention device 100 into a bone hole 140 of a bone. The woven retention device 100 can include a pair of opposing lattice segments, a connecting edge joining the pair of lattice segments on at least a portion of at least one edge, and a bone interface on an exterior surface of one of the lattice segments. The step of inserting the pair of opposing lattice segments can cover only a portion of the circumferential area of the bone hole.

The method can include securing a fastening device 110 into the bone hole 140 having the inserted woven retention device 100. Upon securing the fastening device 110 into the bone hole 140, a portion of the fastening device can interface directly with the surface of the bone in the bone hole and a portion of the fastening device can interface directly with the bone interface of the woven retention device 100.

The method can further include inserting a plurality of woven retention devices 100 into the bone hole 140.

The opposing lattice segments of the inserted woven retention device can provide resistance to rotation of the inserted woven retention device in contact with the fastening device.

The woven retention device can be inserted to just past a distal cortex region 152 of the bone, the woven retention device can be inserted to the distal cortex 152 of the bone, and the fastening device 110 can be secured at a point that corresponds to a start of the insertion at the proximal cortex.

Upon the fastening device being secured, the woven retention device 100 can cover about ⅓ to about ⅛ of the circumference of the bone of the bone hole.

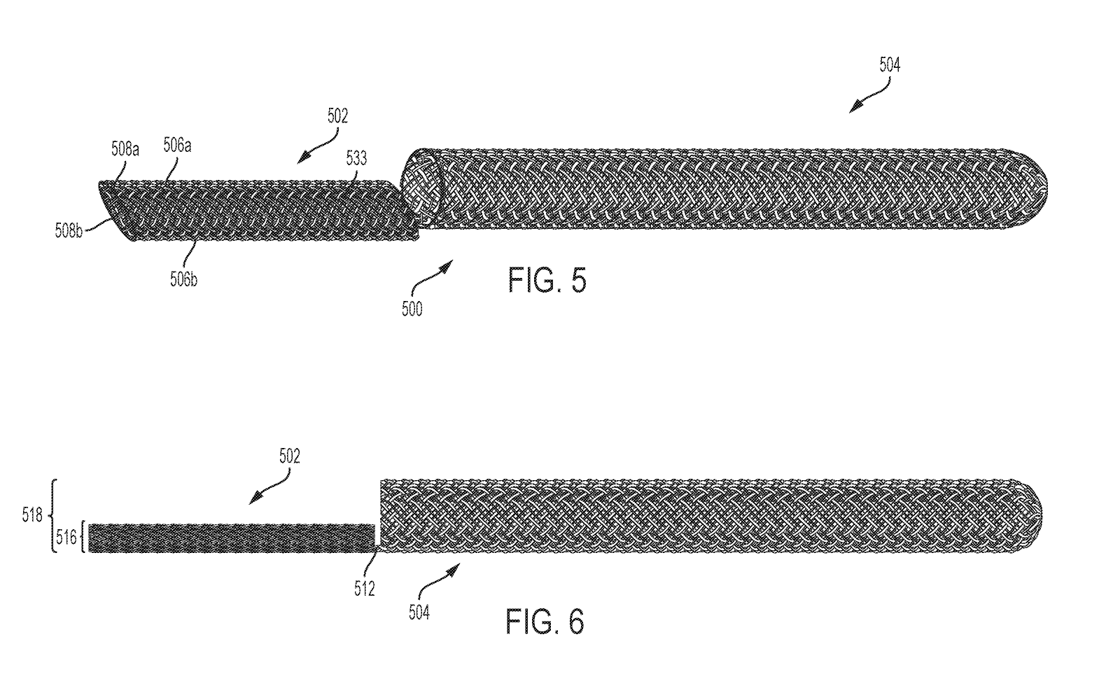

In an embodiment shown in FIG. 5, a proximal portion 502 of the woven retention device 500 can be flattened and a distal portion 504 of the woven retention device 500 can be cylindrical. The fastening device can interface with an exterior surface 507 of the proximal portion 502 and the fastening device can interface with an interior surface 509 of the distal portion 504. Thus, FIG. 5 shows an embodiment of the invention where the distal portion 504 is in a cylindrical state, mating with the distal end of a fastening device along its full circumference. In this embodiment the proximal portion 502 is flattened, mating with a partial part of the screw circumference. FIG. 5 illustrates a closed end 503, which can be useful in bicortical applications. The cylinder closed end may be open at the distal end and/or the open cylindrical end may be placed more proximally.

Advantages of having a cylindrical portion can include: 1) Ease of insertion into the hole; 2) Coverage of distal end of screw when extends past distal end of distal cortex; and 3) Easier entry of screw into proximal end of distal cortex.

The screw can be inserted into or adhered to the two-portioned woven and inserted into the hole by having a cylinder woven be partially transected, after which a screw can be inserted into the lumen of the distal portion of the cylinder and to the side of the flattened proximal portion of the woven retention device.

As seen in FIG. 5, the proximal portion 502 can be flattened, which can result in folded edges or bends 508a, 508b on opposing sides. With this configuration, the folded edges define two lattice segments 508a, 508b. As seen from the figure, lattice segment 508a can be disposed on top of and opposing lattice segment 508b.

FIG. 6 is a side view of an embodiment as shown in FIG. 5. In FIG. 6, the proximal portion 502 of the woven retention device 500 is seen in a flattened state while the distal portion 504 is seen in a cylindrical state. Thus, a height 516 of the proximal portion 502 can be less than a height 518 of the distal portion 504 from the side view. The proximal portion 502 can be connected to the distal portion 504 by a bridge or tab 512.

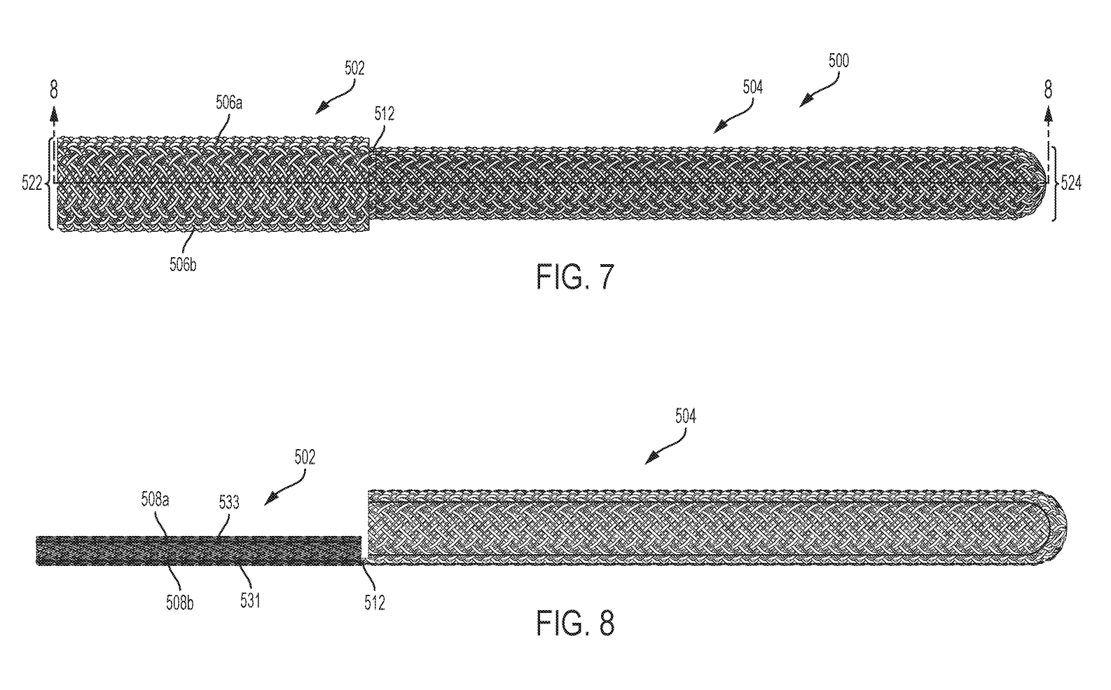

FIG. 7 shows an elevated view of the woven retention device of FIGS. 5 and 6. In FIG. 7, because the proximal portion 502 is flattened and the distal portion 504 maintains the cylindrical shape, the proximal portion can have a width 522 that is greater than a width of the distal portion 524. As can be seen, bridge 512 connects the proximal portion 502 with the distal portion 504. FIG. 7 illustrates that edges 506a, 506b of the proximal portion can be disposed at the periphery of the proximal portion and can comprise folds or bends of the cylindrical material.

FIG. 8 shows a cross-sectional view of FIG. 6. In FIG. 8, top lattice segment 508a and bottom lattice segment 508b of the proximal portion are shown opposing each other. FIG. 8 shows that the bridge 512 connects the proximal portion 502 and distal portion 504.

A method of inserting a screw, comprising: inserting a woven retention device through a pilot hole of a bone, the woven retention device having a lattice that is flattened or configured to be flattened or that has a substantially smaller diameter than the diameter of the hole; and inserting the screw into the pilot hole of the bone, wherein the screw makes direct contact to a surface of the pilot hole of the bone and the flattened surface of the woven retention device.

A woven retention device, comprising: a fixation sleeve, comprising: a substantially tubular lattice of intersecting fibers in a first state, the tubular lattice being configured to be compressed into a flattened lattice inside a pilot hole of a bone, the flattened lattice including a proximal end and a distal end, the proximal end having a receiving portion that is configured to receive a fastener along a longitudinal axis of the fixation sleeve, wherein: the flattened lattice includes an inner surface that has a distributed interface with protruding and recessed portions that are configured to interact with an outer surface of the fastener, the tubular lattice includes an outer surface that has protruding and recessed multiple points of contact configured to interact with an interior bone surface, and the tubular and flattened lattices have a degree of stability that maintains a three-dimensional structure of the tubular lattice and has a degree of flexibility, the degree of stability and flexibility allowing for the distributed interface of the surfaces to distribute applied pressure to the protruding and recessed multiple points of contact of the outer surface, the pressure resulting from the fastener being inserted.

The flattened lattice dynamically micro-loads the fastener into the bone hole by applying pressure from an interfacing surface of the fastener to an interior surface of the woven retention device and by distributing pressure from a fastener being inserted into the woven retention device from a surface of the woven retention device to an exterior surface of the woven retention device for transmission of pressure to bone surface of the bone hole according to a function of bone density and according to a function of an interfacing surface shape of the fastener.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

We claim:

1. A woven retention device comprising:
a pair of connected opposing lattice segments comprising intersecting filaments,
wherein the pair of connected opposing lattice segments have a size and the size is configured for the opposing lattice segments to be placed in a portion of a circumference of a bone hole.

2. The woven retention device of claim 1, wherein the pair of opposing lattice segments are connected at a connecting edge joining the pair of lattice segments on at least a portion of at least one edge.

3. The woven retention device of claim 1, further comprising:
a bone interface on an exterior surface of one of the lattice segments that is configured to interface with a bone surface of a bone hole; and
a fastening device interface on an exterior surface of an other of the lattice segments that is configured to interface with a fastening device.

4. The woven retention device of claim 3, wherein the lattice of intersecting filaments comprises braid patterns that vary protrusion locations of the bone interface and create an asymmetrical pattern to engage a surface of the bone hole.

5. The woven retention device of claim 3, wherein when the woven retention device is inserted into the bone hole, the woven retention device covers an area substantially less than the circumference of the bone hole.

6. The woven retention device of claim 5, wherein the area is about 1/3 to about 1/8 of the circumference of the bone hole.

7. The woven retention device of claim 3, wherein the fastening device is a screw and wherein the other of the lattice segments is formed of a material that does not cut when interfaced with the screw.

8. The woven retention device of claim 7, wherein the pair of opposing segments is lubricious and compliant to embed into bone and adapted to allow the screw to slide past/thread without cutting the lattice segments.

9. The woven retention device of claim 3, wherein the bone interface comprises a plurality of protuberances that are configured to engage with the bone.

10. The woven retention device of claim 1, wherein the pair of opposing lattice segments comprises a continuous lattice of intersecting fibers.

11. The woven retention device of claim 10, wherein the pair of connected opposing lattice segments comprises a flattened woven sleeve.

12. The woven retention device of claim 10, wherein the woven retention device comprises two folds in the continuous lattice.

13. The woven retention device of claim 1, wherein the pair of opposing lattice segments comprises two distinct layers that are coupled on at least one end.

14. The woven retention device of claim 1, wherein a proximal portion of the woven retention device is flattened and a distal portion of the woven retention device is cylindrical,
wherein the fastening device interfaces with an exterior surface of the proximal portion and wherein the fastening device interfaces with an interior surface of the distal portion.

15. The woven retention device of claim 1, wherein at least one of the lattice segments is configured to slide with respect to the corresponding opposing lattice segment.

16. A method of using a woven retention device, comprising:
inserting a woven retention device into a bone hole of a bone, the woven retention device comprising a pair of opposing lattice segments, a connecting edge joining the pair of lattice segments on at least a portion of at least one edge, and a bone interface on an exterior surface of one of the lattice segments, wherein inserting the pair of opposing lattice segments covers only a portion of a circumferential area of the bone hole; and
securing a fastening device into the bone hole having the inserted woven retention device, wherein upon securing the fastening device into the bone hole, a portion of the fastening device interfaces directly with the surface of the bone in the bone hole and a portion of the fastening device interfaces directly with the bone interface of the woven retention device.

17. The method of claim 16, further comprising inserting a plurality of woven retention devices into the bone hole.

18. The method of claim 16, wherein the opposing lattice segments of the inserted woven retention device provide resistance to rotation of the inserted woven retention device in contact with the fastening device.

19. The method of claim 16, wherein the woven retention device is inserted to just past a distal cortex region of the bone, and the fastening device is secured at a point that corresponds to a start of the insertion at a proximal cortex of the bone.

20. The method of claim 16, wherein upon the fastening device being secured, the woven retention device covers an area substantially less than an area of a circumference of the bone hole.

21. The method of claim 20, wherein the area is about ⅓ to about ⅛ of the circumference of the bone hole.

22. The method of claim 16, wherein the woven retention device includes a distal portion that is cylindrical and a proximal portion that is flattened, wherein inserting the fastening device comprises inserting the fastening device into a cylindrical area of the distal portion and abutting the fastening device against the exterior surface of the proximal portion of the woven retention device.
